(19) United States
(12) Patent Application Publication
(10) Pub. No.: US 2026/0108266 A1
(43) Pub. Date: Apr. 23, 2026

(54) SURGICAL INSTRUMENT HAVING A COUPLING DEVICE

(71) Applicant: KARL STORZ SE & Co. KG, Tuttlingen (DE)

(72) Inventors: Janosz SCHNEIDER, Tuttlingen (DE); Daniel KÄRCHER, Tuttlingen (DE); Robin MERZ, Tuttlingen (DE); Sven SCHNEIDER, Tuttlingen (DE); Tobias UNGER, Tuttlingen (DE); Dominik LÄNGLE, Tuttlingen (DE); Judith PÖNISCH, Tuttlingen (DE)

(21) Appl. No.: 19/115,915

(22) PCT Filed: Sep. 29, 2023
(86) PCT No.: PCT/EP2023/077012

§ 371 Date: Mar. 27, 2025

(30) Foreign Application Priority Data
Sep. 29, 2022 (DE) 10 2022 125 154.3

(51) Int. Cl. A61B 17/28; A61B 17/00

(52) U.S. Cl. CPC A61B 17/2841; A61B 2017/00367; A61B 2017/0046; A61B 2017/00477

(57) ABSTRACT

A surgical instrument has a hollow shaft defining a longitudinal axis, a handle arranged at the proximal end of the shaft, and a force-transmission element mounted in the shaft and connected to a tool on the distal side and to the handle on the proximal side. The surgical instrument has a coupling device for releasably connecting the shaft and handle, the coupling device being an engaging device with an engaging element that moves between an engaging position wherein it engages with at least one recess at the proximal end of the shaft and a release position wherein it does not engage with the recess. The engaging element has at least one wedge-like engaging section with an engaging plane, the length corresponding to a length of the recess along the longitudinal axis so that the engaging section is received in the recess up to the engaging plane in the engaging position.

### CROSS REFERENCE TO RELATED APPLICATIONS

This application is a United States National Phase Application of International Application PCT/EP2023/077012, filed Sep. 29, 2023, and claims the benefit of priority under 35 U.S. C. § 119 of German Application 10 2022 125 154.3, filed Sep. 29, 2022, the entire contents of which are incorporated herein by reference.

### TECHNICAL FIELD

The invention relates to a surgical instrument having a coupling device for releasably connecting its shaft to a handle.

### BACKGROUND

Medical or surgical instruments are known from the prior art which have a hollow shaft, a handle arranged at the proximal end of the shaft, and a force transmission element mounted in the shaft, at the distal end of which a tool is arranged which can be controlled by actuating the handle via the force transmission element. A handle is, for example, a handgrip with at least one movable and one immovable handle part. To improve the cleanability and sterilizability of the instruments, the shaft and the handle can be releasably connected to each other via a coupling mechanism. Furthermore, the proximal end of the shaft can be mechanically rigidly connected to a rotary handle part which is rotatably mounted on the handle in order to be able to rotate the shaft relative to the force transmission element and the handle.

EP 3 351 191 B1 discloses a locking bracket for coupling a shaft assembly of a pliers attachment to a special handle. The locking bracket is pivotally arranged on the shaft assembly via a sleeve-like bearing piece and has a holding section which can act on the handle with a circumferential collar so that the shaft assembly is pulled against a cone on the handle. Due to the resilient design of the locking bracket, this connection is free of play. However, such a locking bracket is a relatively expensive component, which is also limited in its use due to the increased space required and therefore cannot be used for all types of surgical instruments. For example, an adaptation to a pistol-shaped handle is not easily possible. Furthermore, this coupling method does not allow shaft rotation.

EP 1 196 097 B1 relates to a connection between the handle and the shaft of a surgical instrument, the shaft being fixed in a sleeve-like housing part of the handle by radially movable spherical locking bodies penetrating into recesses in the shaft. A spring-loaded holding means secures the spherical locking bodies in the recesses and holds a displaceable stop element in a release position, which allows the radial movement of the locking bodies outward into the recesses. This construction, which is enclosed in the housing part, requires a high number of parts and has a relatively large space requirement, the large enclosed region representing a challenge for processing with cleaning and sterilization.

The same applies to a coupling known from EP 0 820 256 B1 for releasably connecting a handle to a shaft tube which is connected at the proximal end to a shaft receiving part. Between a rotating sleeve, which is arranged around the shaft receiving part, and the shaft receiving part, an engaging sleeve which is movable in the axial direction is arranged, which receives at least one spherical engaging element which is movable outwards. The rotating sleeve and/or the handle has/have a conical puncture so that the handle is connected to the shaft tube by engaging the engaging element in the puncture. EP 1 191 888 B1 also discloses a coupling of shaft and handle with spherical engaging elements that are positioned between two conical surfaces.

DE 10 2011 001 890 Al discloses a surgical instrument with an instrument shaft, the shaft end of which has a connecting pin protruding radially to the instrument axis for connection to the handle. This is divided by a slot into two springy arms, at the free ends of which two engaging lugs are formed. The housing of the handle has a corresponding through-hole to form a locking engagement with the connecting pin, which is engaged behind by the engaging lugs so that the connection between the handle and the shaft is secured in a direction of force. However, since the pin with the engaging lugs protrudes from the housing of the handle, it is possible for the engaging connection to be accidentally released during use.

From DE 103 57 104 Al an engaging connection is known in which resilient tongues with engaging elements at the free end are provided as engaging elements on the handle or the shaft insert, which can engage with corresponding recesses or projections on the shaft insert or the handle, respectively. An axially displaceable locking sleeve, which, as a locking element, maintains the engagement of the resilient tongues with the recesses or projections in a secured position, is held in the secured position by spring force.

DE 20 2004 019 174 U1 relates to a surgical instrument in which the handle can be connected to the proximal end of the shaft by a resilient snap connection, the shaft having two longitudinal slots at its proximal end. In these, engaging recesses are formed at a transition from a distal section with a constant width to a proximal section that widens towards the proximal end. The handle has a receiving sleeve into which the proximal end of the shaft can be inserted, an engaging pin protruding in the receiving sleeve entering into one of the engaging recesses. However, in order to secure this snap connection, an additional pin is required, which snaps into a transverse depression at the proximal shaft end.

Another coupling mechanism for releasable connection of shaft and handle is known from EP 1 889 578 B1. The coupling mechanism is designed as a combined clamping and engaging device, a conical proximal end of the shaft being clamped in a correspondingly tapered counter-cone of a sleeve which is mounted in an axially displaceable manner in a receiving bore in the handle. In the housing of the handle, an engaging element is mounted transversely to the receiving bore and such that it can be moved against the force of a spring element. The engaging element consists of a locking member with a keyhole-shaped through-hole in cross section for receiving the sleeve with the clamped proximal shaft end. The shaft is secured via two recesses, in each case, in the proximal end of the shaft and two recesses in the sleeve, into which the locking member of the engaging element engages, so that the sleeve and the shaft are fixed against axial displacement in the handle, rotation of the shaft also being prevented. However, this engaging connection is subject to play, since there must always be a certain amount of play between the engaging element and its guide as well as between the engaging element and the recess in the sleeve, in order to ensure functionality in all conceivable tolerance positions.

DE 100 64 623 C1 describes a spring-loaded engaging element for securing a bayonet connection between the distal shaft end and a pliers head at the tool tip against rotation, which engaging element is mounted at the proximal end region of the shaft, in the handle, in a bore running transversely to the shaft. The engaging element has a keyhole-shaped through-hole with an upper round passage region and a lower narrow grip region. The connecting bar has an engaging region with a rotationally asymmetrical, e.g., rectangular, cross section, which is received in the grip region of the engaging element in a rotationally fixed but longitudinally displaceable manner in the assembled state.

### SUMMARY

It is the object of the present invention to provide a surgical instrument with an improved coupling device for releasably connecting shaft and handle.

This object is achieved by a surgical instrument having features according to the invention.

Developments are set out in the description.

According to a first embodiment, a surgical instrument according to the invention has a hollow shaft defining a longitudinal axis A of the surgical instrument, a handle arranged at the proximal end of the shaft, i.e., the end closest to the operator during use, and a force-transmission element mounted in the shaft, which is connected to a tool on the distal side, i.e., the end remote from the operator, and to the handle on the proximal side. In this case, the surgical instrument has a coupling device for releasably connecting the shaft and the handle, the coupling device being configured as an engaging device with an engaging element which can move between an engaging position in which the engaging element engages with at least one recess at the proximal end of the shaft and a release position in which the engaging element does not engage with the recess. According to the invention, the engaging element has at least one wedge-like (wedge-shape) engaging section which has an engaging plane E, the length of which along the longitudinal axis A corresponds to a length of the recess along the longitudinal axis A, so that the wedge-like engaging section is received in the recess up to the engaging plane in the engaging position.

This engaging device of a surgical instrument according to the invention achieves a reduction in the play between the handle and shaft components, since the contour of the wedge-like engaging section of the engaging element compensates for tolerance differences. The term “wedge-like” refers to a shape of the engaging section in which, like a wedge, two side surfaces facing away from each other, which extend from a rectangular base surface, are not parallel but converge towards each other, so that a distance between the side surfaces decreases over the height. However, the engaging element or the engaging section of the engaging element is not limited to the shape of a wedge, in which two side surfaces facing away from each other meet at a common edge starting from the rectangular base surface, so that the wedge is triangular in profile. Alternatively, the shape of the wedge-like engaging section may also have a base shape deviating from the rectangular shape, as long as it has two parallel sides, of which the two side surfaces facing away from each other extend converging towards each other, so that, as with a wedge, the distance between the side surfaces decreases over the height. Furthermore, a “wedge-like” shape can also be a truncated wedge shape, in which the side surfaces do not converge in a common edge and which is created when the tip of a wedge is cut off with the edge. Furthermore, the two converging side surfaces of the wedge-like engaging section can be configured differently with regard to inclination and course. The side surfaces can be configured as flat surfaces with the same inclination, so that the distance between the side surfaces decreases proportionally over the height. In principle, however, it is also conceivable that the two side surfaces have a different inclination and/or the inclination varies over the height, so that at least one side surface can also be stepped or curved. Consequently, it is possible that the inclination of the converging side surfaces of the wedge-like engaging section is not constant over the height, so that the distance between the side surfaces does not decrease proportionally over the height.

In order to allow rotation of the shaft relative to the handle during use of the surgical instrument, the surgical instrument according to the invention can, in a preferred embodiment, have a rotary handle part which is rotatably mounted on the handle about the longitudinal axis A. In this advantageous embodiment, it is provided that the rotary handle part comprises the engaging device for releasably connecting the handle to the shaft. In this case, the rotatability of the shaft is provided by the mounting of the rotary handle part on the handle, so that the locking engagement of the engaging device with the shaft in the rotary handle part does not require rotatability and can therefore be configured to be non-rotatable. In this case, the non-rotatable connection between the shaft and the rotary handle part contributes to reducing the axial play at the interface between the handle and the rotatable shaft.

In a further embodiment of the surgical instrument according to the invention, the rotary handle part, as part of the engaging device, can have a receiving bore which extends along the longitudinal axis A and is configured to receive the proximal end of the shaft. Furthermore, the engaging device can have a guide bore in the rotary handle part, the guide axis B of which runs in the rotary handle part transversely to the receiving bore, preferably at right angles to the longitudinal axis A. In this guide bore, the engaging element is arranged so as to be movable along the guide axis B.

In a preferred embodiment of the surgical instrument according to the invention, the wedge-like engaging section has a trapezoidal profile which, corresponding to a trapezoidal shape, has a longer base side and a shorter upper side parallel thereto, which are connected by the non-parallel side surfaces. In this case, it is provided that the length of the base side is greater and the length of the upper side is smaller than the length of the recess at the proximal shaft end, so that the engaging plane E of the wedge-like engaging section lies in the region between the longer base side and the shorter upper side of the trapezoidal profile.

A further embodiment of the surgical instrument according to the invention provides that the engaging element is arranged in the guide bore such that the longer base side points away from the shaft and the trapezoidal profile lies in a plane spanned by the longitudinal axis A and the guide axis B or runs parallel thereto.

Particularly preferably, in a further embodiment of the surgical instrument according to the invention, the trapezoidal profile of the wedge-like engaging section can have a rectangular trapezoidal shape, with the side surface which represents the trapezoidal leg that runs at right angles to the base side and to the upper side defining a guide side which—like the non-converging side surfaces of the wedge-like engaging element—supports a linear guidance of the engaging element in the guide bore along the guide axis B.

In order to further reduce the play between the handle and the shaft, in a further embodiment of the surgical instrument according to the invention it can be provided that the engaging device has a spring element, via which the engaging element is prestressed in the direction of the engaging position.

In a further embodiment of the surgical instrument according to the invention, such a spring element can be arranged between the base side of the engaging section of the engaging element and a bottom of the guide bore.

In order to transfer the engaging element from the engaging position into the release position for releasing the locking engagement with the shaft, the engaging element can, in a further embodiment of the surgical instrument according to the invention, have an actuating section which can be configured differently depending on the embodiment of the engaging element.

In a preferred embodiment of the surgical instrument according to the invention, the engaging element can have, in addition to the engaging section, a through-hole for the shaft and an actuating portion, the through-bore being arranged between the actuating section and the wedge-like engaging section. The through-hole can, for example, be cylindrical or keyhole-shaped in cross section. Irrespective of the shape of the through-hole, such an engaging element with engaging section, through-hole and actuating section can have a trapezoidal profile overall, i.e., the actuating section can also have a trapezoidal profile that supplements the trapezoidal profile of the engaging section to form the overall trapezoidal shape of the engaging element. Furthermore, a pressure head can be arranged on the actuating section, or the actuating section can be configured as a pressure head, in order to facilitate the pressure actuation of the engaging element along the guide axis B for releasing the locking engagement. In the engaging position, the pressure head can preferably be located in a position in the guide bore in which it can be easily reached by an operator without protruding from the guide bore in order to avoid unintentional release of the locking engagement.

In principle, however, it is also possible that, in an alternative embodiment to an engaging element with a through-hole, the engaging element has an actuating section which extends from the wedge-like engaging section substantially at right angles to the longitudinal axis A and the guide axis B. Such an actuating section can extend in the manner of a slide controller into a guide groove running parallel to the guide bore, so that the engaging element can be actuated to release the locking engagement parallel to the guide bore.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic side view of a partially disassembled surgical instrument according to the invention;

FIG. 2 is a schematic longitudinal sectional detail view of a surgical instrument according to the invention with the engaging device in the engaging position;

FIG. 3 is a schematic longitudinal sectional detail view of the surgical instrument according to the invention from FIG. 2 with the engaging device in the release position;

FIG. 4 is a perspective schematic detail view of the engaging element of the surgical instrument according to the invention from FIGS. 2 and 3;

FIG. 5 is a schematic side view of an engaging element with a pressure head according to another embodiment of the surgical instrument according to the invention;

FIG. 6 is a schematic side view of an engaging element with an alternatively shaped actuating section according to a further embodiment of the surgical instrument according to the invention;

FIG. 7 is a schematic front view of an engaging element with an alternatively shaped through-hole according to a further embodiment of the surgical instrument according to the invention; and

FIG. 8 is a perspective schematic detail view of an engaging element without a through-hole and with an alternatively configured actuating section according to a further embodiment of the surgical instrument according to the invention.

### DESCRIPTION OF PREFERRED EMBODIMENTS

Referring to the drawings, the invention relates to a surgical instrument which is equipped with a coupling device with reduced play for releasably connecting shaft and handle.

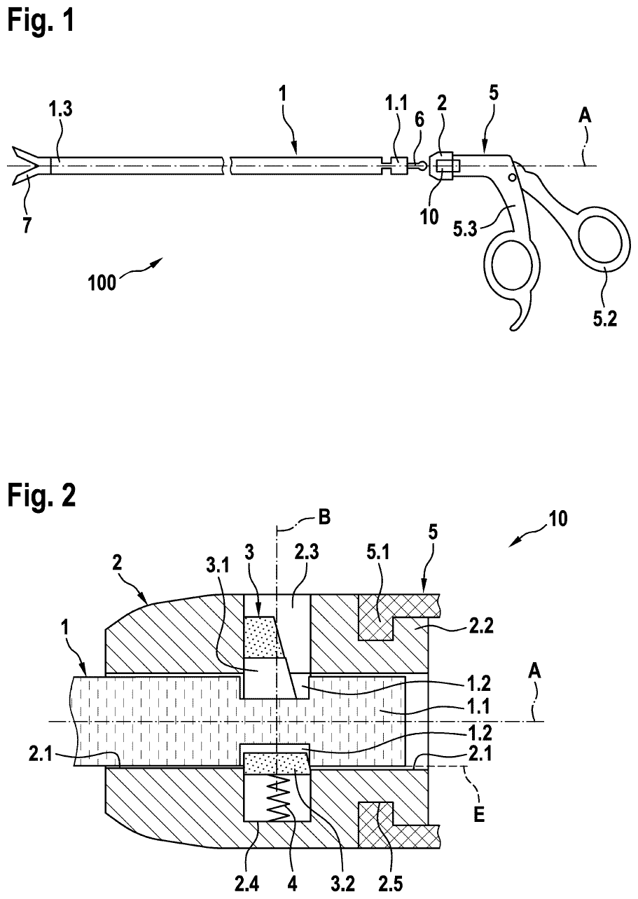

FIG. 1 shows a surgical instrument 100 by way of example, having a hollow outer shaft or shaft 1 defining a longitudinal axis A. At the distal end 1.3 of the shaft 1, a surgical tool 7 is arranged, which is connected to a rod-shaped force transmission element 6, also called a connecting bar, which is mounted in the shaft 1 and extends through the shaft 1. At the proximal end 1.1 of the shaft 1, a handle 5 is arranged, which in this example has an immovable handle part 5.3 and a movable handle part 5.2, which is brought into operative connection with the force transmission element 6 for actuating the tool 7. For releasably connecting the shaft 1 and the handle 5, the surgical instrument 100 according to the invention has an engaging device 10 as a coupling device, which, in the particularly advantageous embodiment shown by way of example in FIG. 1 to 3, is accommodated in a rotary handle part 2, which is arranged on the handle 5 so as to be rotatable about the longitudinal axis A for the rotation of the shaft 1.

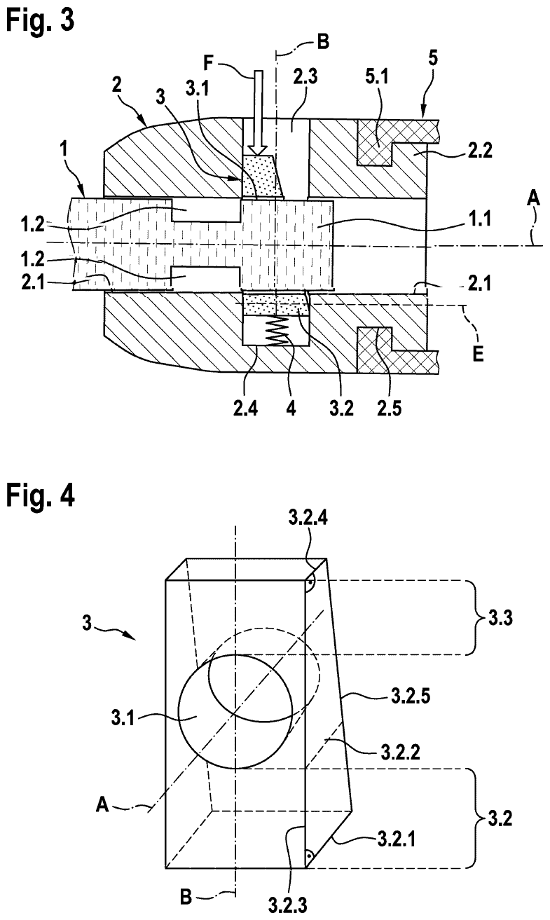

The engaging device has an engaging element 3 for releasably connecting the handle 5 to the proximal end 1.1 of the shaft 1, which engaging element can be moved between an engaging position shown in FIG. 2, in which the engaging element 3 engages with a recess 1.2 at the proximal end 1.1 of the shaft 1, and a release position shown in FIG. 3, in which the engaging element 3 does not engage with the recess 1.2, so that the shaft 1 can be released from the handle 5 or joined thereto. According to the invention, the engaging element 3 has a wedge-like engaging section 3.2 which has a length on an engaging plane E parallel to the longitudinal axis A which corresponds to the length of the recess 1.2 parallel to the longitudinal axis A, so that the engaging section 3.2 is received in the recess 1.2 up to the engaging plane E in the engaging position. In the engaging position, the shaft 1 is fixed against axial displacement in the rotary handle part 2 due to the wedge-like engaging section 3.2 of the engaging element 3, which is rotatably mounted on the handle 5.

The position of the engaging plane E in relation to the height of the engaging section 3.2 and thus the vertical position of the engaging element 3 in relation to the shaft 1 varies depending on the dimensions of the recess 1.2 and the inclination of the wedge-like engaging section 3.2—shown exaggerated in FIG. 2 to 8 for clarity. This advantageously makes it possible to compensate for manufacturing tolerances and thus reduce or even avoid axial play between the connecting partners, shaft and handle 5 or rotary handle part 2.

In order to fix the shaft 3 by means of the engaging element 3, the engaging device 10, in the embodiment shown, has two recesses 1.2 formed diametrically in the proximal end 1.1 of the shaft 1, the engaging element 3 engaging with its engaging section 3.2 in one of the two recesses 1.2 in a locking manner. In principle, only one recess 1.2 at the proximal end 1.1 of the shaft 1 would be required to produce the locking engagement, but the locking engagement can then only take place in a single rotational positioning of the shaft 1 if the circumferential position of the recess 1.2 corresponds to the position of the engaging section 3.2 of the engaging element 3. In the case of two recesses 1.2, the engagement can correspondingly take place in two rotational positions of the shaft 1. Regardless of whether the shaft has one or two or more recesses 1.2, the shaft 1 is not only fixed in the axial direction by the engaging element 3 without play, but is also connected to the rotary handle part 2 in a non-rotatable manner. For this purpose, the width of the engaging element 3 can correspond to the width of the recess 1.2.

In the example of an engaging device 10 of a surgical instrument 100 according to the invention shown in FIGS. 2 and 3, a receiving bore 2.1 which is open on the distal side and runs along the longitudinal axis A is formed in the rotary handle part 2 for receiving the proximal end 1.1 of the shaft 1. The rotary handle part 2 is rotatably mounted on the handle 5, of which only a section is shown in FIGS. 2 and 3, with a radially inwardly projecting collar 5.1, which is in rotatable engagement with a flange section 2.2 of the rotary handle part 2 offset by a neck section 2.5. This schematically simplified view of the rotatable mounting of the rotary handle part 2 on the handle 5 is to be understood only by way of example and is not intended to depict a limitation in any way. Technical solutions for the rotatable mounting of a component such as the rotary handle part 2 on a second component such as the handle 5 are known. For example, the flange section 2.2 can, differently from that shown, be formed separately from the remaining rotary handle part 2 as a threaded ring. The rotary handle part 2 then has a corresponding counter thread on the neck section 2.5. During assembly, the collar 5.1 of the handle 5 is placed on the neck section 2.5, then the threaded ring is screwed on and, after the play has been adjusted, is non-rotatably connected to the neck section 2.5, e.g., by welding or gluing. The engaging element 3, which can be moved between the engaging position and the release position and which is also shown enlarged in FIG. 4, is guided in a guide bore 2.3, the guide axis B of which runs transversely to the receiving bore 2.1, here at right angles to the longitudinal axis A.

In the example shown in FIGS. 2 and 3, the engaging element 3 is operatively connected to a spring element 4 supported on the bottom 2.4 of the guide bore 2.3 in such a way that the engaging element 3 is prestressed in the direction of the engaging position. Thus, the wedge-like engaging section 3.2 is pressed into one of the recesses 1.2 in the engaging position, as shown in FIG. 2. In order to transfer the engaging element 3 into the release position (FIG. 3), an actuating force F directed against the force of the spring element 4 and acting in the direction of the guide axis B, represented by the block arrow, must be applied to the engaging element 3 in order to press the engaging section 3.2 out of the recess 1.2 against the spring force. In the present example with the engaging element 3, which is also shown in FIG. 4, the release position is reached when the shaft 1 is aligned with a cylindrical through-hole 3.1 which is formed in the engaging element 3 between the wedge-like engaging section 3.2 and an actuating section 3.3. This actuating section 3.3 for actuating the engaging element 3 by means of pressure from above extends along or parallel to the guide axis B. The concentric position of the longitudinal axis A with the through-hole 3.1, shown in FIG. 4, therefore refers to the release position of the engaging element 3. In the engaging position, the position of the longitudinal axis A in relation to the engaging element 3 would be further down, e.g., in the lower region of the through-hole 3.1.

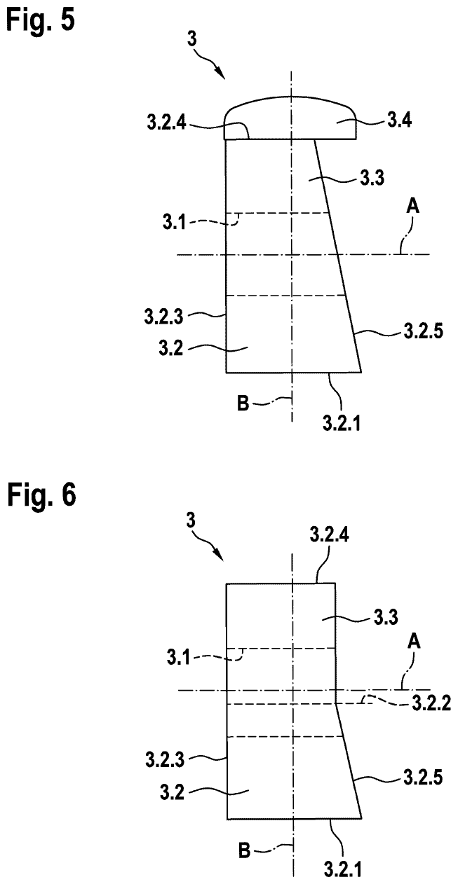

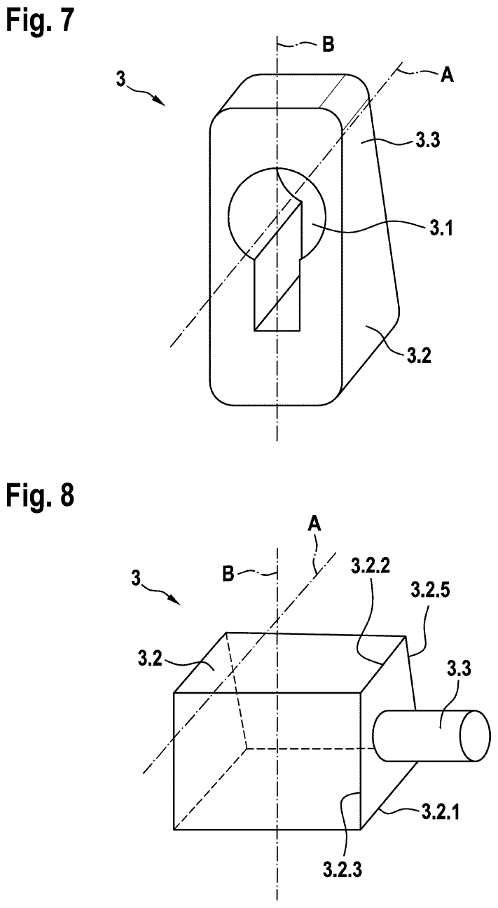

The same also applies to the variants of an engaging element 3 shown in FIGS. 5 and 6, which also have a cylindrical through-hole 3.1 for the shaft 1. The variant of an engaging element 3 in FIG. 7 also has a through-hole 3.1, which, however, has a keyhole-shaped cross section with an upper round region and a lower narrow region. The upper round region of the keyhole-shaped through-hole 3.1 allows the passage of the shaft 1 for assembling or disassembling the instrument 100 when the engaging element 3 is arranged in the release position. As soon as the recesses 1.2 formed in the proximal end 1.1 of the shaft 1 enter the through-hole 3.1, the engaging element 3 is pressed upwards into the engaging position by the spring element 4, the proximal shaft end 1.1 entering the lower narrow region of the keyhole-shaped through-hole 3.1 at the height of the recess 1.2. The variant of the engaging element 3 shown in FIG. 8, however, has an actuating section 3.3 but no through-hole 3.1. This alternative actuating section 3.3 extends there perpendicular to the guide axis B (as well as perpendicular to the longitudinal axis A) from the wedge-like engaging section 3.2, so that an actuation of the engaging element 3 to release the locking engagement can take place parallel to the guide bore 2.3 when the actuating section 3.3 extends through a parallel guide groove (not shown).

In each variant, the engaging element 3 has the wedge-like engaging section 3.2, which allows play-free engagement with the shaft 1. In principle, a wedge is understood to be a body in which two side surfaces converge at an acute angle, but a wedge-like shape is preferably understood to be a prismatic body whose geometric base is a trapezoid, preferably a right-angled trapezoid. In other words, the wedge-like engaging section 3.2 has a trapezoidal profile, the engaging element 3 being arranged in the guide bore 2.3 such that the trapezoidal profile lies in a plane spanned by the longitudinal axis A and the guide axis B or runs parallel thereto.

The trapezoidal profile of the engaging section 3.2 is particularly clearly visible in the side views of FIGS. 5 and 6—however, the following explanations also apply, with appropriate adaptations, to the further embodiments shown in FIGS. 2 to 4, 7 and 8. The trapezoidal profile of the engaging section 3.2 is defined by a longer parallel side as the base side 3.2.1 and a shorter parallel side as the head side 3.2.2, the base side 3.2.1 being longer than the recess 1.2 in the proximal end 1.1 of the shaft 1 along the longitudinal axis A. The head side 3.2.2 is shorter than the recess 1.2, so that the engaging plane E (cf. FIGS. 2 and 3), the length of which corresponds to the length of the recess 1.2, lies between the base side 3.2.1 and the head side 3.2.2 of the wedge-like engaging section 3.2.

In the case of the engaging element 3 in FIG. 5 and also FIGS. 2 to 4 and 7, not only does the engaging section 3.2 have a trapezoidal profile, but also the actuating section 3.3, so that the engaging element 3, apart from a pressure head 3.4 possibly placed on the head side 3.2.4, is also trapezoidal in profile overall. Although the engaging element 3 from FIG. 6 also has an engaging section 3.2 with a trapezoidal profile, the actuating section 3.3, on the other hand, has a rectangular profile so that the head side 3.2.4 can also serve as a pressure head. In the examples shown, the trapezoidal profile of the wedge-like engaging section 3.2 has a rectangular trapezoidal shape, but, in contrast to the examples shown, the engaging section may have a profile that deviates from the rectangular trapezoidal shape. Due to the preferred rectangular trapezoidal shape, a side surface is advantageously provided as the guide side 3.2.3, which is defined by the trapezoidal leg running at right angles to the base side 3.2.1 and the head side 3.2.2. The guide side 3.2.3 supports the linear guidance of the engaging element 3 in the guide bore 2.3. The other side surface is defined by the trapezoidal leg not running at right angles, as an inclined side 3.2.5, which provides the adaptability of the engaging plane E to the length of the recess 1.2.

In principle, the arrangement according to the invention of a wedge-like engaging element 3 for axially fixing a shaft 1 is not limited to the rotary handle part 2. Alternatively thereto, in a surgical instrument according to the invention, an engaging device can also be arranged in the handle itself if, for example, no rotation of the shaft relative to the handle is intended. Such a surgical instrument, also according to the invention, is not shown, but can be transferred correspondingly from the examples with the rotary handle part: In order to arrange the wedge-like engaging element for the play-free axial fixing of the shaft in the handle, the receiving bore for receiving the proximal shaft end is provided in the housing of the handle or a sleeve arranged there. Accordingly, the guide bore for the movable wedge-like engaging element is also formed in the housing of the handle.

The present invention relates to a surgical instrument 100 comprising a hollow shaft 1 defining a longitudinal axis A, a handle 5 arranged at the proximal end 1.1 of the shaft 1, and a force-transmission element 6 mounted in the shaft 1, which is connected to a tool 7 on the distal side and to the handle 5 on the proximal side. In this case, the surgical instrument 100 has a coupling device for releasably connecting of the shaft 1 and the handle 5, the coupling device being an engaging device 10 with an engaging element 3 which can move between an engaging position in which the engaging element 3 engages with at least one recess 1.2 at the proximal end 1.1 of the shaft 1, and a release position in which the engaging element 3 does not engage with the recess 1.2. The engaging element 3 has at least one wedge-like engaging section 3.2 with an engaging plane E, the length of which corresponds to a length of the recess 1.2 along the longitudinal axis A, so that the engaging section 3.2 is received in the recess 1.2 up to the engaging plane E in the engaging position. The drawings, the description, and the claims contain numerous features in combination. A person skilled in the art will also, expediently, consider the features individually and combine them into useful further combinations.

While specific embodiments of the invention have been shown and described in detail to illustrate the application of the principles of the invention, it will be understood that the invention may be embodied otherwise without departing from such principles.

### LIST OF REFERENCE SIGNS

1 shaft
1.1 proximal end
1.2 recess
1.3 distal end
2 rotary handle part
2.1 receiving bore
2.2 flange section
2.3 guide bore
2.4 bottom of the guide bore
2.5 neck section
3 engaging element
3.1 through-hole
3.2 engaging section
3.2.1 base side
3.2.2 upper side
3.2.3 guide side
3.2.4 head side
3.2.5 inclined side
3.3 actuating section
3.4 pressure head
4 spring element
5 handle
5.1 collar
6 actuating element
7 tool
10 engaging device
100 surgical instrument
A longitudinal axis
B guide axis
E engaging plane
F actuating force

1. A surgical instrument comprising;
a hollow shaft defining a longitudinal axis;
a handle arranged at the proximal end of the shaft; and
a force-transmission element mounted in the shaft and connected to a tool on the distal side and to the handle on the proximal side; and
a coupling device for releasably connecting the shaft and the handle and the coupling device being an engaging device with an engaging element which can move between an engaging position in which the engaging element engages with at least one recess at the proximal end of the shaft and a release position in which the engaging element does not engage with the recess wherein the engaging element has at least one wedge-shape engaging section with an engaging plane, the length of which corresponds to a length of the recess along the longitudinal axis so that the engaging section is received in the recess up to the engaging plane in the engaging position.

2. The surgical instrument according to claim 1, wherein a rotary handle part is mounted on the handle so as to be rotatable about the longitudinal axis the rotary handle part comprising the engaging device for releasably connecting the handle to the shaft.

3. The surgical instrument according to claim 2, wherein the engaging device of the rotary handle part comprises:
a receiving bore which extends along the longitudinal axis and is configured to receive the proximal end of the shaft; and
a guide bore whose guide axis in the rotary handle part runs transversely to the receiving bore, the engaging element being arranged in the guide bore so as to be movable along the guide axis.

4. The surgical instrument according to claim 1, wherein the wedge-shape engaging section has a trapezoidal profile with a longer base side and a shorter upper side parallel thereto, a length of the base side being greater than the length of the recess and a length of the upper side being smaller than the length of the recess so that the engaging plane lies between the base side and the upper side of the wedge-shape engaging section.

5. The surgical instrument according to claim 4, wherein the trapezoidal profile of the wedge-shape engaging section has a rectangular trapezoidal shape, a trapezoidal leg that extends at right angles to the base side and the upper side defining a guide side.

6. The surgical instrument according to claim 4, wherein the engaging element is arranged in the guide bore such that the base side points away from the shaft and the trapezoidal profile lies in a plane spanned by the longitudinal axis and the guide axis or runs parallel thereto.

7. The surgical instrument according to claim 1, wherein the engaging device has a spring element via which the engaging element is prestressed in the direction of the engaging position.

8. The surgical instrument according to claim 7, wherein the spring element is arranged between the base side of the engaging section of the engaging element and a bottom of the guide bore.

9. The surgical instrument according to claim 1, wherein the engaging element has a through-hole for the shaft and an actuating section the through-hole being arranged between the actuating section and the wedge-shape engaging section.

10. The surgical instrument according to claim 3, wherein the engaging element has an actuating section which extends from the wedge-shape engaging section substantially at right angles to the longitudinal axis and the guide axis.

11. The surgical instrument according to claim 3, wherein the rotary handle part runs transversely to the receiving bore at right angles to the longitudinal axis.

12. The surgical instrument according to claim 9, wherein the through-hole is cylindrical or keyhole-shaped in cross section, and/or the engaging element has a trapezoidal profile, and/or a pressure head is arranged on the actuating section or the actuating section being configured as a pressure head.
